(19) United States
(12) Patent Application Publication
(10) Pub. No.: US 2019/0309243 A1
(43) Pub. Date: Oct. 10, 2019

(54) CONTAINER AND BIOGAS INSTALLATION

(71) Applicant: BIOENERGY CONCEPT GMBH, Lüneburg (DE)

(72) Inventor: Christoph Eusterbrock, Deutsch Evern (DE)

(21) Appl. No.: 16/334,709

(22) PCT Filed: Sep. 19, 2017
(86) PCT No.: PCT/EP2017/073674

§ 371 Date: May 14, 2019

(30) Foreign Application Priority Data
Sep. 20, 2016 (DE) 10 2016 218 051.7

(51) Int. Cl. C12M 1/107; C12M 1/00

(52) U.S. Cl. CPC C12M 21/04; C12M 23/18

(57) ABSTRACT

So as to specify a container (2), in particular for generating biogas, comprising a preferably cylindrical main portion (3) and a base portion (9) arranged below the main portion (3), and a biogas system, which are of a simple, cost-effective construction that makes simple maintenance possible and which, with low energy consumption, effectively prevent blockages and funnel formation, it is proposed for the base portion (9) to have an annularly closed circulation duct (18).

### CROSS-REFERENCE TO RELATED APPLICATIONS

This application is a US National Stage Entry Under 35 USC § 371 of International Patent Application No. PCT/EP2017/073674, entitled “CONTAINER AND BIOGAS INSTALLATION,” naming as inventor Christoph Eusterbrock, and filed Sep. 19, 2017, which claims priority to German Patent Application No. 102016218051.7, filed Sep. 20, 2016, which applications are hereby incorporated herein by reference in their entireties.

The present invention relates to a container, in particular for generating biogas, comprising a preferably cylindrical main portion and a base portion arranged below the main portion.

The invention further relates to a biogas system.

Containers of the type mentioned at the outset are known for example as components of biogas systems of various configurations and various sizes. Often, the container is configured as a steel tank or reinforced concrete tank and has a volume of 400-6000 m³. The biomass provided for biogas generation is filled into the container in the form of liquid sludge. This liquid sludge is a suspension, and so there is the problem of depositions. In conventional containers in biogas systems, the base is formed substantially planar. In practice, for the previously known containers, it is therefore often a problem in biogas systems that blockages and a deposition of heavy materials on the base occur, as a result of sinking of the heavy components of the liquid sludge to be treated.

To address this topic, a device for anaerobic decomposition of sludge is previously known from DE 42 08 148 A1, and is conically configured or has an egg shape in a lower housing region. In the prior art, an important effect of the conical or egg-shaped base cone is to provide a transition zone for moving decomposition liquid contained in the digester to or out of the housing base and the lower end of the suction pipe. Therefore, in the prior art, the inclination of the conical housing should be selected so as to ensure that the decomposing sludge is not deposited and thus isolated from the mixing process. However, a drawback of the container previously known from DE 4 208 148 A1 is that the problem of blockages and funnel formations due to sludge depositions persists in practice and that the investment costs for this embodiment are very high.

In general, therefore, in conventional containers for generating biogas large-sized stirring units are used so as effectively to prevent blockages and sedimentations. Disadvantageously, however, the equipment requirements, the investment costs and the operating costs due to high energy expenditure are considerable.

Against the described background, the object of the present invention is to specify a container of the type mentioned at the outset and a biogas system which are of a simple, cost-effective construction, which makes simple maintenance possible and, with low energy consumption, effectively prevents blockages and sedimentation on the container base.

According to the invention, the object relating to the container is achieved by a container, in particular for generating biogas, comprising a preferably cylindrical main portion and a base portion arranged below the main portion, in which the base portion has an annularly closed circulation duct. Because an annular duct for passing a circulation flow around the vertical axis of the container is arranged on the base of the container, depositions in the base region of the container which lead to blockages and sedimentation can be effectively prevented. In this way, a stirring unit in the main portion of the container can advantageously be sized much smaller or in the most favourable case be omitted entirely, without having to accept blockages. Advantageously, both the energy consumption of the system and other operating costs can be reduced by comparison with conventional containers.

Preferably, in one embodiment of the invention, the base portion has a substantially planar central region, which is horizontally delimited by the circulation duct. A central planar base portion is thus advantageously enclosed by a circulation duct. This circulation duct may for example be configured as an annularly closed gutter, the base of which is lower than the planar central region of the base portion. As a result of the circulation in the circulation duct, according to the invention depositions in the planar central region are advantageously prevented if circulation is generated in the circulation duct by suitable means.

In particular, in an advantageous embodiment of the container according to the invention, the circulation duct has an inlet opening having an inlet cross section so as to admit medium into the circulation duct substantially tangentially to the vertical axis of the container. In this way, as a result of the supply of medium through the inlet opening, circulation can be generated in the circulation duct and advantageously counteracts deposition of solids in the base region.

It is likewise preferred if, in the container according to the invention, the circulation duct has an outlet opening having an outlet cross section so as to discharge medium from the circulation duct substantially tangentially to the vertical axis of the container. For example, a pump may be connected to the outlet opening so as to suction medium out of the container along the circulation duct. This results in annular circulation in the circulation duct, which can advantageously prevent depositions in the base region. Advantageously, to achieve this effect, it is not necessary to provide energy-intensive stirring units in the main portion of the container, of which the stirring power is sufficient to bring about revolutions even in the base region. According to the invention, any stirring unit that may be provided in the main portion can thus be configured much smaller. A biogas system comprising a container of this type can thus advantageously be operated energy-efficiently and thus cost-effectively.

If, preferably, a bypass line connecting the outlet opening to the inlet opening is provided, a circulating flow, based in whole or in part on recirculation of medium discharged from the container, can be brought about in the circulation duct. In particular, part of the medium emptied out of the circulation duct via the outlet opening can be supplied to a store for storing solid residues from the biogas generation. On the other hand, via the bypass line, a partial flow can be branched off from the pumped-out flow and recirculated into the circulation channel via the inlet opening.

In an advantageous embodiment of the container according to the invention, an inlet pump for admitting medium into the circulation duct via the inlet opening and/or an outlet pump for discharging medium from the circulation duct via the outlet opening are provided. Advantageously, in this way, in the context of the invention circulation can be built up in the circulation duct by using a pump. If a separate inlet pump and a separate outlet pump are provided, the ratio between recirculated medium and discharged medium can be set by controlling the power of the inlet pump and outlet pump relative to one another. On the other hand, in the context of the invention, a single pump may also be used, so long as a bypass line is provided which connects the outlet opening to the inlet opening.

In this connection, it is favourable if means for varying the cross section of the bypass line are provided. For example, according to the invention, the variation means may be configured as a slider, which opens the cross section of the bypass line to a greater or lesser extent. In this way, if a pump is used in the circulation system, the ratio of medium pumped out to the store to medium recirculated into the circulation duct can be set continuously during operation.

To set a ratio between recirculated and discharged medium, in the context of the invention it is favourable if the outlet cross section is configured larger than the inlet cross section. In this case, a larger amount of sediment-containing sludge is discharged from the circulation duct and a smaller amount is recirculated into the circulation duct via the inlet, so long as the inlet opening and the outlet opening communicate via a bypass line.

In practice, it has been found to be particularly appropriate for the diameter of the outlet opening to be approximately twice as large as the diameter of the inlet opening.

In one embodiment of the invention, it has been found to be advantageous if the circulation duct is made from a plastics material and preferably cast together with the central region. The circularly closed circulation duct may be produced and shaped cost-effectively in particular by using polyethylene. The polyethylene annular duct can thus be cast together with the central region, which is configured for example as a reinforced concrete base.

In a development of the container according to the invention, a portion which is tapered, preferably conically, towards the base portion is connected to the base portion in the direction of the vertical axis of the container. According to the invention, this for example conically tapered region has in particular a type of funnel shape. The funnel shape advantageously brings about tapering, in other words reduction, of the cross section of the container towards the base. In practice, the container may have for example a diameter of 30 m in the region of the preferably cylindrical main portion. By contrast, according to the invention, it has been found to be favourable if the planar central region of the base portion has a diameter of merely approximately 3 m-6 m. As a result of the tapered container portion according to the invention, the container cross section is uniformly reduced from a relatively large cross-sectional area in the region of the main portion to a much smaller diameter in the region of the central region. On the one hand, this is advantageous for counteracting deposition of sediment in the base region as a result of the flow conditions if a stirring unit is operated in the main portion. On the other hand, it has been found that, for the effect of a circulation duct for preventing depositions in the base portion, a planar central region of the base portion must not have too large a diameter. The tapered container portion, in particular conical portion, thus in effect acts as an adapter between the main portion of the container and the base portion.

Meanwhile, in the context of the invention, in principle the main portion of the container according to the invention may also preferably be of a conically tapered configuration.

Alternatively, in the context of the invention, the conically tapered container portion may be arranged between the base portion and the main portion. So as to make it possible to produce the, in particular conically, tapered portion according to the invention particularly cost-effectively, the tapered portion has plastics material walls. PE-HD has been found to be suitable as a material, in particular even when groundwater protection aspects are taken into account. This material is tried and tested for example in embodiments of biogas systems which are configured as lagoons, in other words as film-lined earth basins.

So as additionally to counteract sedimentation of sinking materials on the base of the container, according to the invention, stirring units for stirring the medium located in the container may also be provided in the container in the main portion. Advantageously, however, as a result of the embodiment according to the invention of the container, comprising an annularly closed circulation duct in the base portion, the stirring units can be sized much smaller. As a result, the energy intake of a biogas system is significantly reduced if a container according to the invention is used.

The above object is equally achieved according to the invention by a biogas system which has a container according to any of claims 1 to 13, of which at least a base portion is introduced into the ground, a gravel bed being provided below the base portion. By way of the gravel bed, in particular any water protection regulations that may be in place can be adhered to. Since the gravel bed only has to underpin the base region, which as a result of the configuration only has a comparatively small diameter, the production thereof is much more favourable than for conventional containers.

In a preferred variant of the biogas system, it is particularly favourable if the tapered portion is also introduced into the ground. The lower portion of the container is accordingly configured in the manner of a lagoon, in other words a film-lined earth basin. However, as a result of the base portion configured according to the invention, comprising an annularly closed circulation duct, the drawbacks which occur in conventional lagoons as regards preventing sediment deposition can advantageously be prevented.

By way of example, the invention is described in a preferred embodiment with reference to the drawings, further advantageous details being deducible from the drawings.

Functionally equivalent parts are provided with like reference numerals.

### BRIEF DESCRIPTION OF THE DRAWINGS

In the drawings, in detail:

FIG. 1 is a vertical section through a biogas system according to the invention in a preferred embodiment comprising a container according to the invention in a preferred embodiment;

FIG. 2 is a detailed view of the region II of FIG. 1, showing the base region of the biogas system of FIG. 1;

FIG. 3 is a schematic plan view of a preferred embodiment of a base portion of a container according to the invention.

### DETAILED DESCRIPTION

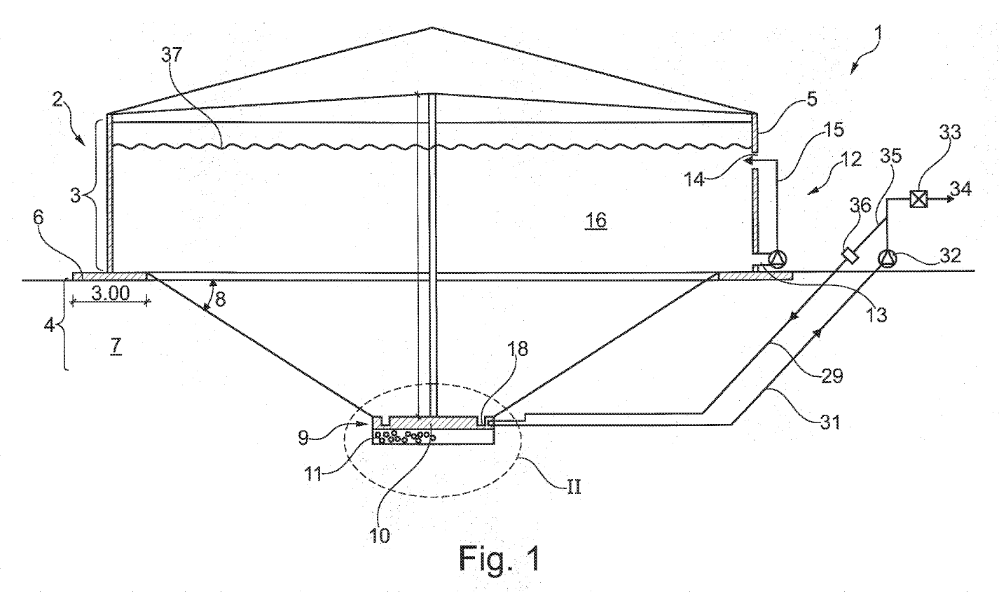

FIG. 1 is a vertical sectional view of a preferred embodiment of a biogas system 1 according to the invention comprising a container 2 in an embodiment of the invention. As can be seen in FIG. 1, the container 2 has a cylindrical main portion 3, to which a conically tapering portion 4 is connected downwards. The cylindrical main portion 3 has a wall 5 of reinforced concrete. The wall 5 is formed on an annular plinth foundation 6.

The conically tapered portion 4 is located in the ground 7 in the manner of a lagoon. The tapered portion 4 is made from PE-HD. The wall of the tapered portion 4 is at an angle 8 of 10°-45°, preferably 30°, to the horizontal.

The tapered portion 4 is terminated downwards by a base portion 9. The base portion 9 substantially consists of a reinforced concrete base plate 10. The base plate 10 is mounted on a gravel bed 11 provided in the ground 7 in the region of the base plate 10, so as to pump out groundwater if required.

In the embodiment presently being described, the diameter of the cylindrical main portion 3 is approximately 24 m, whilst the diameter of the base plate 10 according to the example is approximately 5 m. The conically tapered portion 4 therefore has a vertical extent of approximately 6 m, depending on the angle 8.

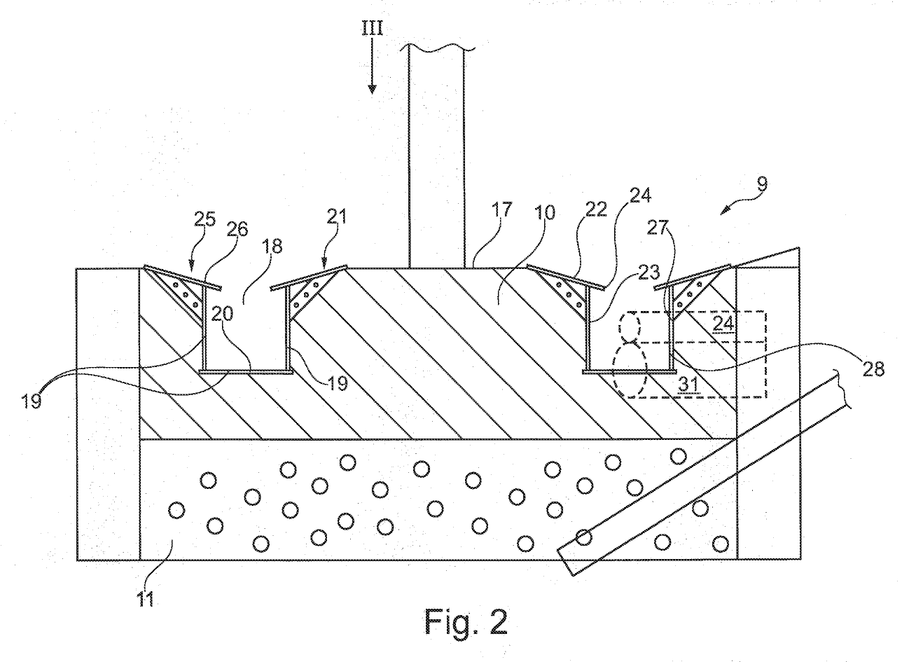

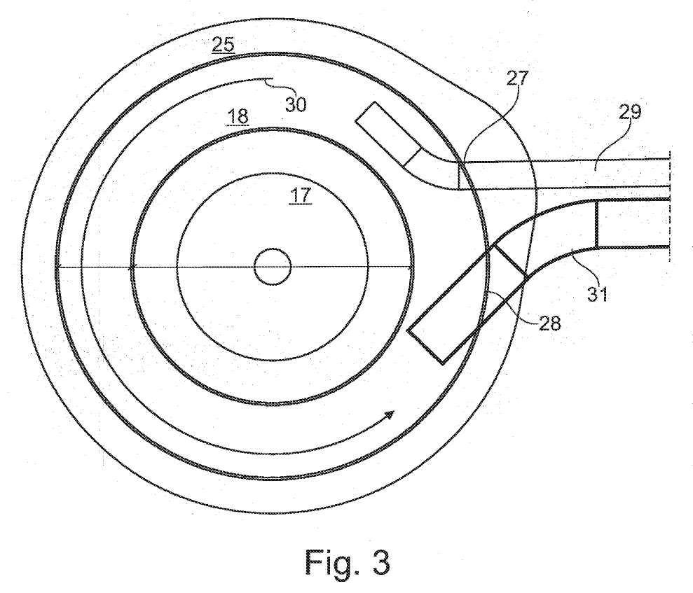

The wall 5 of the main portion 3 of the container 2 according to the invention is provided with a stirring system 12. The stirring system 12 substantially consists of a lower opening 13 in the wall 5 and an upper opening 14 in the wall 5 as well as a pipeline 15 connecting the lower opening 13 to the upper opening 14. A pump is arranged in the pipeline 15 so as to withdraw medium 16, stored in the cylindrical main portion 3 of the container 2, from the lower opening 13 and recirculate it into the container 2 via the upper opening 14. In this way, a revolution can be generated in such a way that nutrients contained in the media 16 are supplied to the active bacteria as uniformly as possible. Hereinafter, the base portion 9 of the container 2 of the biogas system 1 of FIG. 1 is explained in greater detail with reference to FIGS. 2 and 3. In this connection, FIG. 2 is an enlargement of the detail II of FIG. 1. As can be seen in FIG. 2, the reinforced concrete base plate 10 has a substantially planar central region 17. The planar central region 17 is annularly enclosed by an annular duct 18. The annular duct 18 consists of polyethylene plates 19, which are cast together with the reinforced concrete base plate 10. The base 20 of the annular duct 18 is below the planar central region 17 of the base plate 10.

In the transition between the planar central region 17 of the base plate 10 and the annular duct 18, there is an annular transition region 21 at an inclination to the horizontal. In the embodiment shown here, the inclined transition region 21 is lined with a polyethylene plate 22. The polyethylene plate 22 overhangs the vertical inner wall 23 of the annular duct 18 at the radially outer end 24, in the manner of a roof. Moreover, in the radially outer region, the base plate 10 has a further radially inwardly inclined region 25, which itself annularly encloses the annular duct 18. Like the inclined transition region 21, the inclined outer region 25 is lined with polyethylene plates 26, which protrude into the annular duct 18 in the manner of a roof.

FIG. 3 is a plan view of the base portion 9 viewed in the direction of the arrow III in FIG. 2. As can be seen particularly clearly in FIG. 3, the annular duct 18 has an inlet opening 27 and an outlet opening 28. A polyethylene inlet pipe 29 is passed through the inlet opening 27 in such a way that, when medium is supplied through the inlet pipe 29 in a tangential direction, an annular flow 30 is generated in an anticlockwise direction.

On the other side, an outlet pipe 31, through which medium can be withdrawn from the annular duct 18 in a tangential direction so as to generate or reinforce the annular flow 30, is passed through the outlet opening 28.

Returning again to FIG. 1, hereinafter the effect of the inlet pipe 29 and the outlet pipe 31 for generating the annular flow 30 in the annular duct 18 is explained in greater detail. As can be seen in FIG. 1, the outlet pipe 31 is connected to a pump 32, which communicates via a valve 33 with a storage container 34 (not shown in greater detail). It can further be seen that a bypass line 35, which closes together the outlet pipe 31 and the inlet pipe 29, is provided at the output of the pump 32. It can further be seen that a slider 36 (only schematically shown) is provided in the bypass line 35. By means of the slider 36, the ratio of the medium conveyed by the pump 32 and recirculated into the annular duct 18 through the inlet opening 27 via the bypass line 35 and the inlet pipe 29 to the proportion of medium passed into the storage container 34 can be set.

Incidentally, further components of the biogas system 1, for example for withdrawing the generated biogas, are not shown in greater detail in FIG. 1. These may be configured in a conventional manner well known to an appropriate person skilled in the art.

For operating the biogas system 1 comprising the container 2 of FIGS. 1 to 3 according to the invention, medium 16 is filled into the container 2 to a fill level 37 via an opening (not shown in FIG. 1). The medium 16 is biomass as a starting material for the biogas generation. Sludges having a high proportion of heavy material/sinking material, for example of sand or grit, are also conceivable as a medium 16. It is initially the stirring system 12, which ensures revolution of the medium 16 in the container 2, that ensures that the bacteria are supplied with organic nutrients and kept suspended, in particular for large diameters of the main portion 3, for example in the region of 10-40 m.

At the same time, however, according to the invention the annular flow 30 in the annular duct 18 in the base plate 10 serves to prevent encrusting and funnel formation in the base portion 9 of the container 2. For this purpose, by means of the pump 32, sludge is suctioned in the annular duct 18 via the outlet pipe 31 and the outlet opening 28, and can be supplied to a storage container via the valve 33. However, so as to maintain an annular flow 30 in the annular duct 18, part of the sludge suctioned via the outlet pipe 31 is recirculated into the annular duct 18 through the inlet opening 27 via the bypass line 35 and the inlet pipe 29. The ratio of this recirculation flow can be set by way of the slider 36 (not shown in greater detail) in the bypass line 35.

The slider 36 may be configured in any desired manner known to a person skilled in the art, for example as a control valve.

As a result of the annular flow 30 in the annular duct 18, prevention of depositions of sinking materials in the base portion 9 of the container 2 is brought about with a comparatively low energy input. Therefore, revolution by way of the stirring system 12 in the cylindrical main portion 3 of the container 2 can be operated with comparatively low energy expenditure without problems occurring.

Because the conically tapered portion 4 comprising polyethylene walls is arranged between the cylindrical main portion 3 having a large diameter, for example 15 m or more, and the base portion 9, the base portion 9 has a much smaller diameter, for example 3 m, than the main portion 3.

As regards setting the proportion of medium recirculated through the inlet pipe 29 via the bypass line 35, according to the invention a presetting is provided in that the diameter of the inlet pipe 29 is selected smaller than the diameter of the outlet pipe 31. It has been found to be particularly favourable if the diameter of the inlet pipe 29 is 110 mm and the diameter of the outlet pipe 31 is approximately 200 mm.

In this way, a container and a biogas system are disclosed which can be operated with lower investment costs and lower operating costs than conventional systems and containers, without blockages and sedimentations occurring as a result of sinking materials.

### LIST OF REFERENCE NUMERALS

1 Biogas system
2 Container
3 Main portion
4 Conically tapered portion
5 Wall
6 Plinth foundation
7 Ground
8 Angle
9 Base portion
10 Base plate
11 Gravel bed
12 Stirring system
13 Lower opening
14 Upper opening
15 Pipeline
16 Medium
17 Planar central region
18 Annular duct
19 PE plates
20 Base
21 Inclined transition region
22 PE plate
23 Inner wall
24 Outer end
25 Inclined outer region
26 PE plate
27 Inlet opening
28 Outlet opening
29 Inlet pipe
30 Annular flow
31 Outlet pipe
32 Pump
33 Valve
34 Storage container
35 Bypass line
36 Slider
37 Fill level

What is claimed is:

1. A container for generating biogas comprising:
a cylindrical main portion;
a base portion arranged below the cylindrical main portion, characterised in that the base portion has an annularly closed circulation duct;
the circulation duct having an inlet opening having an inlet cross section so as to admit medium into the circulation duct substantially tangentially to a vertical axis of the container, the circulation duct having an outlet opening having an outlet cross section so as to discharge medium from the circulation duct substantially tangentially to the vertical axis of the container; and
a bypass line for connecting the outlet opening to the inlet opening, wherein the bypass line comprises a cross section that is variable.

2. The container according to claim 1, characterised in that the base portion has a substantially planar central region which is horizontally delimited by the circulation duct.

3. The container according to claim 1, further comprising an inlet pump for admitting medium into the circulation duct via the inlet opening and/or an outlet pump for discharging medium from the circulation duct via the outlet opening.

4. The container according to claim 1, characterised in that the outlet cross section is configured larger than the inlet cross section.

5. The container according to claim 1, characterised in that the circulation duct is made from a plastics material and cast together with a central region.

6. The container according to claim 1, characterised in that a tapered portion, which is shaped conically towards the base portion, is connected to the base portion in a direction of the vertical axis of the container.

7. The container according to claim 6, characterised in that the tapered portion has plastics material walls.

8. The container according to claim 6, wherein, the tapered portion is introduced in ground in a film-lined earth basin.

9. The container according to claim 1, further comprising a stirring system for stirring medium located in the container.

10. The container, according to claim 9, wherein the stirring system comprises a lower opening, an upper opening and a pipeline.

11. The container, according to claim 1, wherein the base portion is introduced in ground on top of a gravel bed.

12. A container for generating biogas comprising:
a cylindrical main portion;
a base portion arranged below the cylindrical main portion, characterised in that the base portion has an annularly closed circulation duct;
the circulation duct having an inlet opening having an inlet cross section so as to admit medium into the circulation duct substantially tangentially to a vertical axis of the container, the circulation duct having an outlet opening having an outlet cross section so as to discharge medium from the circulation duct substantially tangentially to the vertical axis of the container; and
a stirring system for stirring medium located in the container.

13. The container according to claim 12, further comprising a bypass line for connecting the outlet opening to the inlet opening, wherein the bypass line comprises a cross section that is variable.

14. The container according to claim 12, characterised in that the base portion has a substantially planar central region which is horizontally delimited by the circulation duct.

15. The container according to claim 12, further comprising an inlet pump for admitting medium into the circulation duct via the inlet opening and/or an outlet pump for discharging medium from the circulation duct via the outlet opening.

16. The container, according to claim 12, wherein the stirring system comprises a lower opening, an upper opening and a pipeline.

17. A container for generating biogas comprising:
a cylindrical main portion;
a base portion arranged below the cylindrical main portion, characterised in that the base portion has an annularly closed circulation duct;
the circulation duct having an inlet opening having an inlet cross section so as to admit medium into the circulation duct substantially tangentially to a vertical axis of the container, the circulation duct having an outlet opening having an outlet cross section so as to discharge medium from the circulation duct substantially tangentially to the vertical axis of the container; and
an inlet pump for admitting medium into the circulation duct via the inlet opening and/or an outlet pump for discharging medium from the circulation duct via the outlet opening.

18. The container according to claim 17, comprising a stirring system for stirring medium located in the container, wherein the stirring system comprises a lower opening, an upper opening and a pipeline.

19. The container according to claim 17, characterised in that a tapered portion, which is shaped conically towards the base portion, is connected to the base portion in a direction of the vertical axis of the container.

20. The container according to claim 17, characterised in that the outlet cross section is configured larger than the inlet cross section.
